(19) United States
(12) Patent Application Publication
(10) Pub. No.: US 2005/0010140 A1
(43) Pub. Date: Jan. 13, 2005

(54) Shockwave or pressure-wave type therapeutic apparatus

(75) Inventor: Bernd Forssmann, Herrsching (DE)

Correspondence Address: KING & SPALDING LLP, 191 PEACHTREE STREET, N.E., ATLANTA, GA, 30303-1763, US

(73) Assignee: Dornier MedTech Systems GmbH, Wessling (DE)

(21) Appl. No.: 10/857,162

(22) Filed: May 28, 2004

Related U.S. Application Data
(63) Continuation of application No. PCT/DE02/04351, filed on Nov. 27, 2002.

(30) Foreign Application Priority Data
Nov. 29, 2001 (DE) 101 58 519.5

(51) Int. Cl. A61B 17/22

(52) U.S. Cl. 601/4

(57) ABSTRACT

A shockwave or pressure-wave therapeutic apparatus is provided in which a therapy head is equipped with a shockwave source. The shockwave source is connected via a shock generator to a control means which controls the release frequency of shockwaves as a function of the pulse energy thereof in such a manner that higher pulse energies correlate with lower release frequencies of the shockwaves and vice versa.

### CROSS-REFERENCE TO RELATED APPLICATION

This application is a continuation, under 35 U.S.C. § 365(a), of the co-pending PCT patent application having International Application No. PCT/DE02/04351, having International Filing Date 27 Nov. 2002 (27.11.2002) and Priority Date 29 Nov. 2001 (29.11.2001), which claims priority to German Patent Application No. 101 58 519.5, filed on Nov. 29, 2001, and which is incorporated herein by reference. Additionally, this application claims priority under 35 U.S.C. § 119(a) to German Patent Application No. 101 58 519.5 filed on Nov. 29, 2001, and which is incorporated herein by reference.

### FIELD OF THE INVENTION

The invention generally relates to the application of ultrasonic energy to biological tissue, and more particularly, to a shockwave or pressure-wave type therapeutic apparatus.

### BACKGROUND OF THE INVENTION

It sometimes happens that when passing through the tissue of living beings, shockwaves produce a sufficiently known disadvantageous result in the form of the formation of bubbles. Said bubbles are created as a direct consequence of the interaction of the shockwave with the coupling liquid or the tissue. The duration of said bubbles is in general short. However, cavitation nuclei and long-life bubbles that intensify the formation of bubbles of a subsequent shockwave may also be created. This phenomenon is designated by the experts as cavitation in the broadest sense.

Cavitation is disadvantageous in two respects, namely during the destruction of concrements, which lasts much longer due to cavitation and is disadvantageous for the patient for this reason alone. In the meantime it was found during research work in this field that the cavitation in the shockwave path increases with the intensity of the shockwave and the frequency with which the shockwaves follow one another. The absorption of the respectively successive shockwaves in the cavitation field leads, on the one hand, to a reduction of the fragmentation of the concrements and, on the other hand, to an increase in the side effects on the patient, above all in the form of painful tissue impairments.

These problems were e.g. already pointed out in 1998, namely in a scientific treatise by Peter Huber et al., published in “Phys. Med. Biol. 43 (1998) 3113-3128. Printed in UK”. Furthermore, it is known from an article by Ryan Paterson et al., published in the “Journal Of Urology”, Vol. 165, No. 5, Supplement, Jun. 6, 2001” that the efficiency of stone destruction is associated with decreasing the pulse frequency in shockwave lithotripsy. Finally, H. Wiksell and A. C. Kinn already concluded in 1995 that with an increasing shockwave release frequency in lithotripsy the efficiency thereof is decreasing.

### SUMMARY OF THE INVENTION

It is an object of the present invention to avoid, as much as possible, the above-mentioned drawbacks in shockwave or pressure-wave type therapeutic apparatus of the above-mentioned kind, and to provide a means which enables a physician to work as efficiently as possible with the above-mentioned therapeutic apparatus with the disadvantageous impacts on the patient being as small as possible.

### BRIEF DESCRIPTION OF THE DRAWINGS

The accompanying drawings are incorporated into and form a part of the specification for the purpose of explaining the principles of the invention. The drawings are not to be construed as limiting the invention to only the illustrated and described examples of how the invention can be made and used. Further features and advantages will become apparent from the following and more particular description of the invention which is illustrated in the accompanying drawings, wherein:

FIGS. 1 to 3 show three exemplary block diagrams illustrating the connection of a control means to a shock generator of a shockwave source in accordance with exemplary embodiments of the present invention.

### DETAILED DESCRIPTION OF THE INVENTION

Exemplary embodiments of the present invention will be described hereinafter with reference to the drawings wherein like elements and structures are indicated by like reference numbers. In accordance with such exemplary embodiments, a shock generator, e.g., of an above-mentioned therapeutic apparatus, that supplies the electrical energy for the shockwave or pressure wave source is connected to a control means which controls the release frequency of the shockwaves or pressure waves, hereinafter only called shockwaves, as a function of the pulse energy thereof in such a manner that higher pulse energies correlate with lower release frequencies of the shockwaves, and vice versa. The energy applied per time unit can be fixed to a critical limit value by coupling the release frequency with the shockwave energy; this limit value can not or must not be exceeded. A high pulse energy determines the low release frequency, and higher release frequencies can be chosen for lower pulse energies.

A coupling between shock generator and control means can be fixed according to hardware, software or software/hardware, i.e. in an obligatory or optional manner. For instance, the user may be bound by prerequisites or he is only informed that in a given case he is leaving the critical range. In practice, the critical values are empirically determined values. Scientifically speaking, it may be useful to determine said critical value from the so-called half-life period, where the half-life period is the period after which the intensity of the cavitation bubble has decreased to half the amount. The critical value may be lowered in an extreme case to such an extent that an interaction between successive shockwaves can be ruled out.

For controlling the intensity of the cavitation bubbles, it is possible, via a measuring device, e.g. via a measurement of light scattering or absorption on the cavitation bubbles, to measure the intensity thereof in the shockwave field of the coupling liquid or the transmission medium of the shockwave to choose the measurement values according to the invention as a reference for the release frequency. It is useful to correlate said measurement values also with the half-life period; the theoretical half-life period can here serve as a threshold value. In this connection it is possible to use a high-resolution ultrasound in the manner known per se to measure in vivo the cavitation as to its decrease intensity in the shockwave path, e.g. in kidney parenchyma.

It is e.g. also known that shockwaves introduced into the human body may affect cardiac activity by extrasystoly. In this case the shockwaves should be released in ECG-triggered manner. It is of advantage when the ECG signal from the ECG is processed for shockwave release also in the control means as additional information such that, when the ECG release frequency is above the critical release frequency, the ECG release frequency is reduced in that e.g. every second, third, and so on trigger pulse is ignored so that the desired release frequency is achieved on average. Such a precautionary measure may save human lives under specific circumstances.

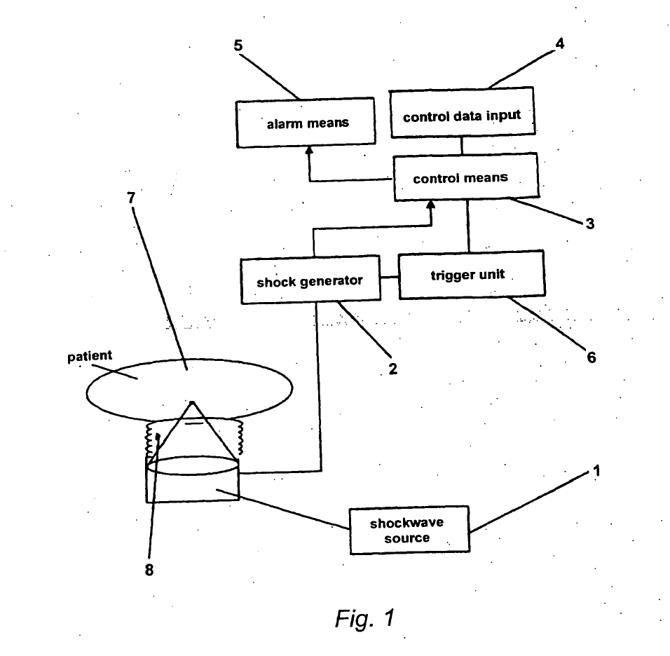

The invention is graphically explained in the figures. FIGS. 1 to 3 show three different block diagrams for illustrating the connection of the control means according to the invention to a shock generator of a shockwave source. According to FIG. 1 a shockwave source 1 is connected via a shock generator 2 to a control means 3 in which control data to be observed can be input as limit values above all with respect to the pulse energy and the release frequencies of the shockwaves via an input means 4. The control means 3 of the invention can thus indicate to the treating physician via an alarm means 5 connected to the control means 3, namely by way of alarm signals, such as flash light, audio signals, or the like, that specific limit values will be reached soon or have already been exceeded.

In principle, the connection of the control means 3 to the shock generator 2 may also be designed such that the limit values for the pulse energy and the release frequencies of the shockwaves are observed automatically and will thus have to be observed by the treating physician by necessity. For reasons of circuitry the shock generator 2 is additionally connected to the control means 3 via a trigger unit. A coupling bellows 8 is positioned between the patient 7 and the shockwave source 1.

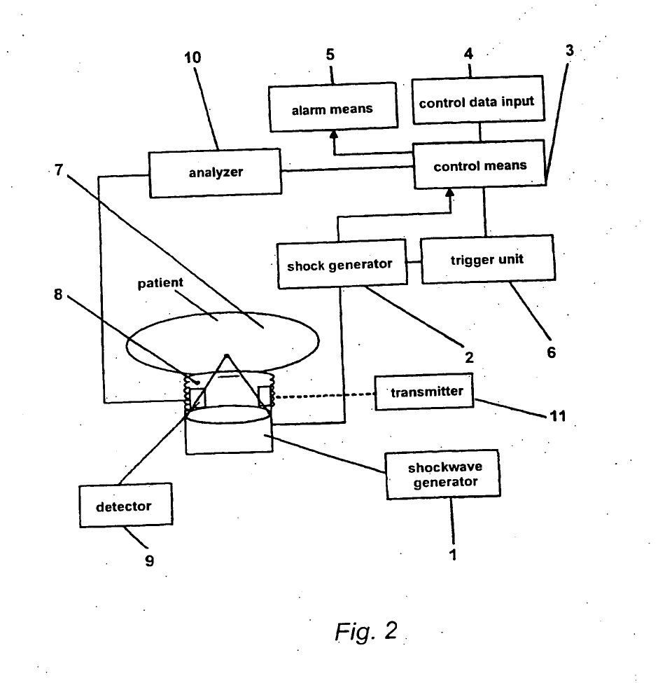

FIG. 2 shows a further block diagram illustrating a possible different integration of the control means 3 of the invention into the surroundings of a shockwave type therapeutic apparatus. The cavitation bubbles are here sensed via a detector 9 in the coupling pad 8 and the corresponding signals are evaluated in an analyzer 10. This kind of determining control data is based on the fact that the strength of the formation of the cavitation bubbles inside the coupling pad 8 between the shockwave source 1 and the patient 7 correlates with the cavitation bubble formation in the body of patient 7.

Additional information can be provided via the input channel 4 of the control means 3, e.g., an algorithm, as to how the analyzed cavitation is to be evaluated and the release frequency is to be controlled. For detecting the cavitation bubbles, the light scattering or absorption can, e.g., be used with the help of a transmitter 11 in the form of a light source and a detector 9. The use of ultrasound would also be suited therefore.

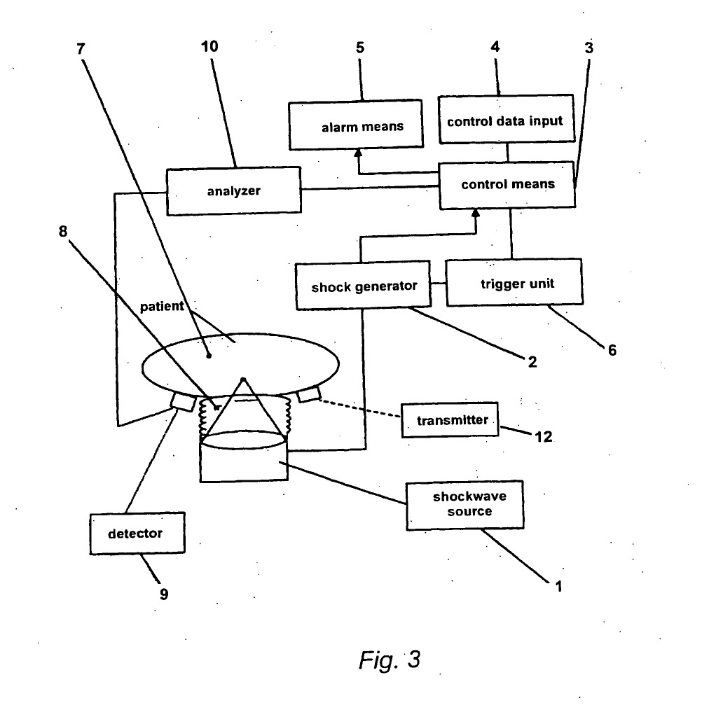

According to the block diagram in FIG. 3 a transmitter 12 and a detector 13 measure the cavitation directly in the body of a patient 7. For this measurement ultrasound is preferably suited. Transmitter 12 and detector (receiver) 13 can advantageously be accommodated in one and the same housing.

The physician may here also be warned via the control means 3 with the help of alarm means 5 as soon as he is within critical ranges. The alarm means 5 may also be used for automatically correlating the release frequency with the pulse energy of the shockwave.

While the invention has been described with respect to the foregoing exemplary embodiments, it will be apparent to those skilled in the art that various modifications, variations and improvements of the invention can be made in light of the above teachings and within the purview of the appended claims without departing from the spirit and intended scope of the invention. In regard to the foregoing description of the exemplary embodiments of the invention, areas which are known to those of ordinary skill in the art have not been described in detail in order to facilitate a clear and concise description of the invention. Accordingly, it should be understood that the invention is not to be limited by the specific exemplary embodiments, but only by the scope of the appended claims.

1. A shockwave or pressure-wave type therapeutic apparatus, comprising a therapy head equipped with a shockwave source, wherein the shockwave source is connected via a shock generator to a control means which controls a release frequency of shockwaves as a function of a pulse energy thereof so that increased pulse energies correlate with lower release frequencies of the shockwaves and decreased pulse energies correlate with higher release frequencies of the shockwaves.

2. The shockwave or pressure-wave type therapeutic apparatus of claim 1, wherein the correlation between the release frequency and the pulse energy of the shockwaves can be set according to software and/or hardware.

3. The shockwave or pressure-wave type therapeutic apparatus of claim 1, wherein the release frequency of the shockwaves is below a half-life period or a similar critical value.

4. The shockwave or pressure-wave type therapeutic apparatus of claim 1, wherein the control means is connected to an alarm means.

5. The shockwave or pressure-wave type therapeutic apparatus of claim 1, wherein the control means automatically controls the shock generator according to a fixed program according to the limit values entered for pulse energy and release frequency of the shockwaves.

6. The shockwave or pressure-wave type therapeutic apparatus of claim 1, wherein the shockwave source can exclusively be switched on via the control means and is only operative via the control means as long as the control means controls a therapy according to a predetermined correspondence curves of release frequency and pulse energy.

7. The shockwave or pressure-wave type therapeutic apparatus of claim 1, further comprising a detector arranged in a coupling pad connected to the shockwave source, wherein the detector corresponds with a transmitter for checking the intensity of cavitation bubbles in a coupling liquid or in a transfer medium and the detector passes on detected signals via an analyzer to the control means.

8. The shockwave or pressure-wave type therapeutic apparatus of claim 7, wherein the transmitter is designed as a light source so that light scattering and/or absorption of the cavitation bubbles is detected by the detector and can be passed on via the analyzer to the control means.

9. The shockwave or pressure-wave type therapeutic apparatus of claim 8, wherein the transmitter is designed as an ultrasonic generator and the detected signals are passed on to the control means.

10. The shockwave or pressure-wave type therapeutic apparatus of claim 1, wherein an intensity of cavitation bubbles is detected by means of a transmitter or a detector directly in a body of a patient and is passed on via an analyzer to the control means.

11. The shockwave or pressure-wave type therapeutic apparatus of claim 10, wherein the transmitter is designed as an ultrasonic generator.
